(19) United States
(12) Patent Application Publication
(10) Pub. No.: US 2011/0274679 A1
(43) Pub. Date: Nov. 10, 2011

(54) Compositions and Methods of SIRT Activation

(75) Inventor: Zbigniew Pietrzkowski, Aliso Viejo, CA (US)

(73) Assignee: VDF FutureCeuticals, Inc., Momence, IL (US)

(21) Appl. No.: 13/127,796

(22) PCT Filed: Nov. 5, 2009
(86) PCT No.: PCT/US09/63358

§ 371 Date: Jul. 25, 2011

Related U.S. Application Data
(60) Provisional application No. 61/111,538, filed on Nov. 5, 2008.

(51) Int. Cl. A61K 31/675; C07F 9/58; A61P 43/00; C07D 475/14; A61K 31/525; C12N 5/00; A61K 31/714; C07H 23/00

(52) U.S. Cl. 424/94.1; 514/89; 514/52; 546/24; 536/26.4; 544/251; 514/251; 435/375

(57) ABSTRACT

Compositions and methods of SIRT activation are presented in which one or more vitamin compounds, and especially vitamin B compounds are used to significantly increase SIRT activity in vitro and in vivo. In especially preferred compositions, vitamin B6, vitamin B12, and vitamin B2 are present in synergistic quantities.

This application claims priority to our copending U.S. provisional patent application with the Ser. No. 61/111,538 filed Nov. 5, 2008 which is incorporated by reference herein in its entirety.

### FIELD OF THE INVENTION

The field of the invention is activation of SIRT, especially as it relates to activation of SIRT using nutritional supplements, and vitamins in particular.

### BACKGROUND OF THE INVENTION

Sirtuins are ubiquitously found in animals, plants, and various microorganisms, and are thought to play a key role in an organism's response to various stress signals. On a molecular level, Sirtuins can be characterized as protein (and especially histone) deacetylases that use NAD⁺ as cofactor (EC number 3.5.1). Remarkably, increased SIRT activity has been associated in experimental systems with lifespan-extending effects of calorie restriction, improved glucose/lipid metabolism, and increased PON1 (Paraoxonase 1) activity, which is known as the major anti-atherosclerotic component of high-density lipoprotein.

Not surprisingly, SIRT activation has become an attractive target for pharmaceutical and nutraceutical companies, and numerous platforms to identify SIRT activators have been reported as can be taken from WO 06/081329, U.S. Pat. App. No. 2008/0021063, or U.S. Pat. No. 7,273,713. While such methods are at least conceptually promising, various difficulties still remain. Most significantly, compounds identified using such methods will typically require a full investigation into their pharmacologic, pharmacodynamic, and/or toxicologic profile prior to marketing.

To avoid disadvantages associated with new drug entities, certain naturally occurring compounds have been considered suitable for SIRT activation as they have been reported to stimulate SIRT. Among other compounds, various flavones, stilbenes, catechins, flavanones, and anthocyanidins, were described in U.S. Pat. App. No. 2006/0084135 as SIRT activators. Similarly, resveratrol was demonstrated to activate Sir2 in Saccharomyces cerevisiae (see e.g., Nature 2004; 430(7000): 686-689.), and implications for treatment of various conditions in human and ageing were described elsewhere (see e.g., Clin Intery Aging. 2008; 3(2):331-9). While resveratrol and related natural compounds are generally regarded as safe and readily available from various natural materials, significant quantities are typically required to elicit a measurable effect in vitro. Moreover, resveratrol has relatively poor solubility in water and bioavailability is thus severely restricted, typically producing serum concentrations of resveratrol that are insufficient for significant biological effects in vivo.

Thus, there is still a need to improved compositions and methods of SIRT activation in mammals, and especially with pharmaceutically or nutritionally acceptable compositions.

### SUMMARY OF THE INVENTION

The inventor has discovered that SIRT activity in a mammal, and especially in human can be significantly increased by administration of one or more vitamins, and especially by oral administration of vitamin B compounds, where such administration uses dosages that are ordinarily taken as dietary supplements.

Consequently, a method of providing a composition that increases SIRT activity in a mammal is contemplated that has a step in which a composition is formulated that includes at least one vitamin, and particularly a vitamin B compound in an amount effective to increase SIRT activity in the mammal. In another step, a test result is obtained that indicates that the vitamin B compound increases SIRT activity in the mammal, in yet another step, the composition is provided to the mammal in association with the test result. In especially preferred aspects, the vitamin B compound is vitamin B₆, vitamin B₁₂, and/or vitamin B₂, more preferably vitamin B₆ and vitamin B₁₂, and most preferably vitamin B₆, vitamin B₁₂, and vitamin B₂. Additionally, it is contemplated that the vitamin B₆, vitamin B₁₂, and vitamin B₂ are present in a synergistic amount. Where desired, the composition may further comprise vitamin B₁, vitamin B₅, and/or vitamin B₉. Optionally, contemplated compositions will further include a flavone, a stilbene, a flavanone, and/or an anthocyanidin.

It is further generally preferred that vitamin B₆ is present in an amount of between 60 and 600 mg, vitamin B₁₂ is present in an amount of between 30 and 300 mcg, and that vitamin B₂ is present in an amount of between 30 and 300 mg. Where additional vitamin B compounds are present, it is preferred that vitamin B₁ is present in an amount of between 30 and 300 mg, vitamin B₅ is present in an amount of between 30 and 300 mg, and that vitamin B₉ is present in an amount of between 3 and 30 mg.

Most preferably, vitamin B₆ is present in an amount of about 220 mg, the vitamin B₁₂ is present in an amount of about 90 mcg, vitamin B₂ is present in an amount of about 100 mg, vitamin B₁ is present in an amount of about 100 mg, vitamin B₅ is present in an amount of about 100 mg, and that vitamin B₉ is present in an amount of about 15 mg.

It is still further contemplated that test result is a test result obtained from a human test subject, and/or that the increase SIRT activity is at least 10%, more typically at least 50%, and most typically at least 80% over untreated control.

Therefore, a method of increasing SIRT activity in a mammal is contemplated in which to a mammal is administered a synergistic combination of vitamin B compounds in an amount effective to increase SIRT activity in the mammal. Preferably, the synergistic combination of vitamin B compounds comprises vitamin B₆, vitamin B₁₂, and vitamin B₂, and may further include vitamin B₁, vitamin B₅, and/or vitamin B₉.

In particularly contemplated methods, vitamin B₆ is present in an amount of between 60 and 600 mg, vitamin B₁₂ is present in an amount of between 30 and 300 mcg, and vitamin B₂ is present in an amount of between 30 and 300 mg, and where added, vitamin B₁ is present in an amount of between 30 and 300 mg, vitamin B₅ is present in an amount of between 30 and 300 mg, and/or vitamin B₉ is present in an amount of between 3 and 30 mg. Most preferably, vitamin B₆ is present in an amount of about 220 mg, vitamin B₁₂ is present in an amount of about 90 mcg, vitamin B₂ is present in an amount of about 100 mg, vitamin B₁ is present in an amount of about 100 mg, vitamin B₅ is present in an amount of about 100 mg, and vitamin B₉ is present in an amount of about 15 mg.

Preferably, the vitamin blend comprises a vitamin B₆ compound and a vitamin B₁₂, and most preferably a vitamin B₆ compound, a vitamin B₁₂, and a vitamin B₂ compound, typically at a concentration that induces SIRT activity at least 50%, and most typically at least 100% over an untreated control using an experimental system as described below. In still further contemplated formulations, the blend further comprises a vitamin B₁ compound, a vitamin B₃ compound, a pantothenate compound, and/or a folate compound. Consequently, all methods are contemplated in which a consumer (wholesale and/or retail) is advised that the compositions according to the inventive subject matter are effective to increase SIRT activity in a mammal when administered to the mammal.

Various objects, features, aspects and advantages of the present invention will become more apparent from the following detailed description of preferred embodiments of the invention.

### BRIEF DESCRIPTION OF THE DRAWING

FIG. 1 is a graph illustrating in vitro activation of SIRT using selected compounds and mixtures of compounds.

FIG. 2 is a graph illustrating in vitro activation of SIRT using selected compounds and mixtures of compounds.

FIG. 3 is a graph illustrating in vivo activation of SIRT in peripheral blood samples of human volunteers to which an exemplary mixture of compounds was administered.

### DETAILED DESCRIPTION

The inventor has discovered that SIRT activity in a mammal and mammalian cells can be significantly increased in vitro and in vivo by administration of a vitamin to the mammal, and especially a vitamin B compound. Therefore, in preferred aspects of the inventive subject matter, certain vitamins, and particularly vitamin B compounds are used to increase SIRT activity in a mammal or mammalian cells, and in especially preferred aspects, combinations of certain vitamins, and particularly vitamin B compounds, are used to significantly increase SIRT activity.

For example, the inventor discovered that the unexpected effect of vitamins on SIRT was particularly pronounced with specific combinations of vitamins from the B-group of vitamins, which could be still further enhanced to at least some degree by combination with yet other vitamin B compounds (and especially vitamin B₅ and vitamin B₉) and those related to them. It should be particularly noted that the increase of SIRT activity with one or more of selected vitamin B compounds is especially unexpected as vitamin B₃ is a known inhibitor of SIRT (see e.g., Trends Biochem Sci. 2005 September; 30(9):479-83). Interestingly, the inventor also discovered that while some vitamins per se failed to provide a significant increase in SIRT activity, selected combinations of the same vitamins with other vitamins showed substantial effect on SIRT activity. Such effect is completely unexpected as none of the vitamins appear to be directly associated with restriction in caloric uptake. Moreover, none of the vitamins in combination were previously reported to have a clinically proven effect that was different from their individual effects.

The terms “SIRT” and “sirtuin” are used interchangeably herein and refer to the class of deacetylases that includes SIRT1 to SIRT7, and especially to SIRT1 (which is also known as a human homolog of SIR2L1, Sir2, Sir2a, or Silent mating type Information Regulation-2 protein). As also used herein, the terms “SIRT activation” or “increases SIRT activity” mean that the overall observable catalytic activity of SIRT is increased, which may be caused by various factors, including increased transcription, increased translation, and/or increased catalytic activity (increased specific activity).

Based on the inventor's observations and experimental data (infra), the inventor thus contemplates a method of providing a composition that increases SIRT activity in a mammal in which in one step a composition is formulated that has at least one vitamin B compound in an amount effective to increase SIRT activity in the mammal. In another step, a test result is obtained that indicates that the vitamin B compound(s) increases SIRT activity, and in yet another step, the composition is provided to the mammal in association with the test result.

In a particularly preferred aspect of the inventive subject matter, the composition is a nutritional supplement, snack, drink, or other edible item that includes a combination of vitamin B₆, vitamin B₁₂, and optionally vitamin B₂, preferably in the following amounts: Vitamin B₆ is present in an amount of about 220 mg, vitamin B₁₂ is present in an amount of about 90 mcg, and vitamin B₂ is present in an amount of about 100 mg. As used herein, the term “about” when used in conjunction with a numeral refers to a range +/−10% of the numeral, inclusive. Most notably, as can be seen from the data presented below, such combination is a synergistic combination with respect to SIRT activation. The term “synergistic combination” of vitamin B compounds as used herein refers to combination of the compounds in which the sum of the individual effects of the respective compounds is less than the observed combined effect of the compounds when used together.

Among other suitable combinations, one exemplary SIRT activating combination of B-vitamins was formulated that included a combination of vitamin B₂, B₁, B₃, B₆, B₁₂, and B₅. Using that combination, HeLa cells were separately treated in vitro for 4 hrs, along with a reference compound (here: resveratrol) as positive control, as well as individually with the respective above vitamins at a total concentrations of 0.1% (wt/vol) of culture medium. To measure SIRT activation, the cells were lysed after treatment and SIRT activity was determined using a commercially available SIRT Activity Assay kit (BioMol, Inc.; Catalog #KI-104).

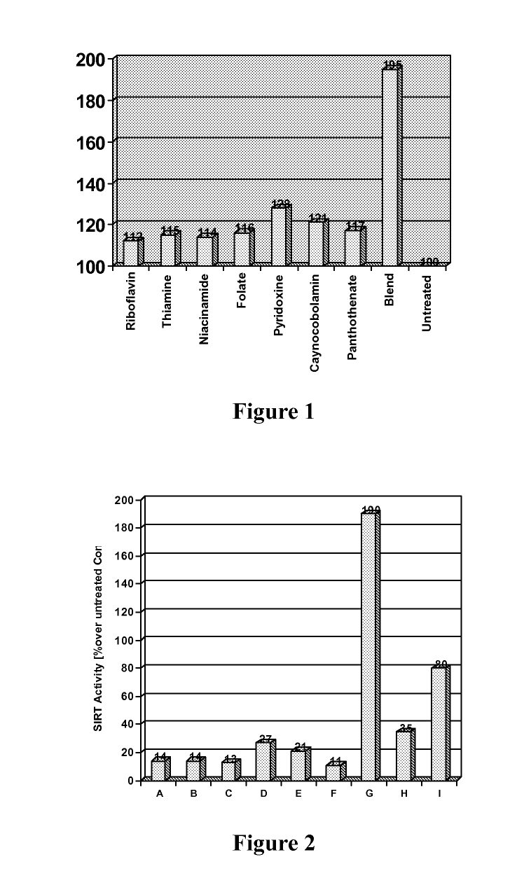

As can be seen from FIG. 1, all vitamins stimulate activity of SIRT, however, at a relatively low level. Remarkably, certain combinations (see below, others not shown) had a strong combined effect that reflects at least an additive, and in some cases synergistic effect on SIRT activity under the same experimental conditions. Moreover, in vitro activation with the known SIRT activator resveratrol was, depending on the cell line, between about 15-120% of untreated control, making the SIRT activation with the vitamin compounds even more remarkable.

In an experiment similar to that above, FIG. 2 depicts further combinations for selected vitamin B compounds in which the columns show the results for A (Riboflavin, vitamin B₂), B (Thiamine, vitamin B₁), C (Niacinamide, vitamin B₃), D (Pyridoxine, vitamin B₆), E (Cyanocobolamin, vitamin B₁₂), F (Ca-Panthotenate, vitamin B₅), G=Blend of A-F (VSIRT), H (Pyrodoxine, vitamin B₆ plus Cobalamin, vitamin B₁₂), 0.05% each, and I (H plus Riboflavin, vitamin B₂) (0.033% each). Here, the stimulatory effect of selected vitamins on SIRT activity in vitro is expressed in percent over untreated control. As is readily apparent, the minimum combination of vitamin B₆ and B₁₂, and the tri-fold combination of B₆, B₁₂, and B₂ have remarkable stimulatory (and synergistic for the tri-fold combination) effect on SIRT activity.

To determine if the specific combinations of the vitamins were also effective in vivo in human, three volunteers were enlisted and 700 mg/2100 mg of vitamin blend G (VSIRT; supra) were administered on empty stomach. Table 1 below depicts the ingredients of the formulation used and the average amount per vitamin expressed in mg (with the exception of B₁₂ where the quantity is expressed in micrograms (mcg)).

TABLE 1

| Compound | Source | mg of components |
|---|---|---|
| Riboflavin (B₂) | Sigma: R7649 | 97.4 |
| Thiamine HCL (B₁) | Sigma: T4625 | 103.8 |
| Niacinamide (B₃) | Sigma: N5535 | 240.0 |
| Pyridoxine HCL (B₆) | Sigma: P9755 | 224.9 |
| Folic Acid (B₉) | Sigma: F7876 | 15.1 |
| Cyanocobalamin (B₁₂) | Sigma: C3607 | 91.6 mcg |
| d-Ca pantothenate (B₅) | Sigma: C8731 | 100.3 |

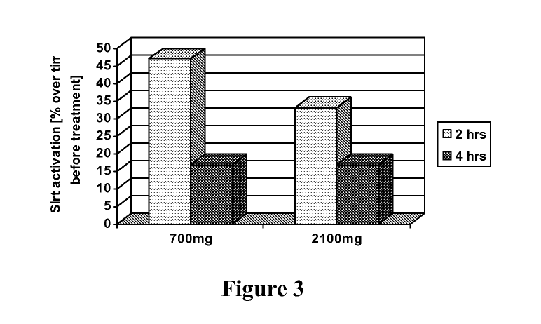

Blood was collected at time 0 before treatment, and 2 and 4 hrs after treatment with 700 mg or 2100 mg of vitamin blend. SIRT activity was measured in peripheral blood cells (PBC) using the same method as described above. PBC were collected from subjects treated with VSIRT for 2 or 4 hrs and at doses of 700 or 2100 mg. Remarkably, contemplated compositions were demonstrated to stimulate SIRT activity up to 47% (average based on data from three subjects) in peripheral blood cells collected after first 2 hours. This effect is reduced during next 2 hrs (total 4 hours of the treatment) to 16% by average as is shown in FIG. 3. Here, each bar represents an average value of data collected from 3 subjects in each dose group.

Of course, it should be appreciated that various alternative dosages and compositions are also deemed suitable for use herein. For example, suitable daily dosages of contemplated compositions will typically be between 10 mg and 5000 mg, more typically between 50 mg and 2000 mg, and most typically between 500 mg and 1500 mg (with respect to total weight of active ingredients in formulation

Therefore, it should be recognized that the combinations contemplated herein will generally include at least two, and more typically three of the compounds of Table 1. For example, where the combination includes at least two compounds, vitamins B₆ and B₁₂ are particularly preferred, and where the combination includes at least three compounds, vitamins B₆, B₁₂, and B₂ are especially preferred. For example, while Table 1 provided particularly preferred average dosages, numerous alternative dosage ranges for the individual components are also expressly contemplated. Table 2 below provides an exemplary list of general and preferred dosage ranges (in mg per day; vitamin B₁₂ is expressed in mcg per day).

TABLE 2

| Compound | Gen. Range (mg) | Preferred Range (mg) |
|---|---|---|
| Riboflavin (B₂) | 10-1000 | 30-300 |
| Thiamine HCL (B₁) | 10-1000 | 30-300 |
| Niacinamide (B₃) | 20-2000 | 60-600 |
| Pyridoxine HCL (B₆) | 20-2000 | 60-600 |
| Folic Acid (B₉) | 1-100 | 3-30 |
| Cyanocobalamin (B₁₂) | 10-1000 mcg | 30-300 mcg |
| d-Ca Pantothenate (B₅) | 10-1000 | 30-300 |

Remarkably, however, it was also observed that addition of further ingredients (e.g., certain minerals and fruit extracts; data not shown) did reduce and in some cases even abolish SIRT activation.

With respect to vitamin B compounds it should be noted that that all vitamins in the B-group of vitamins (and derivatives thereof) are deemed suitable for use herein. Therefore, especially preferred vitamin B compounds include vitamin B₁, vitamin B₂, vitamin B₃, vitamin B₅, vitamin B₆, vitamin B₇, vitamin B₉, and vitamin B₁₂. However, it should be noted that vitamin B3 is typically not preferred, and in some aspects even excluded from the compositions according to the inventive subject matter.

Thus, and among other combinations, especially preferred combinations include those in which vitamin B₁, is combined with at least one (and more typically at least two) vitamin selected from the group of vitamin B₂, vitamin B₃, vitamin B₅, vitamin B₆, vitamin B₇, vitamin B₉, and vitamin B₁₂, those in which vitamin B₂, is combined with at least one (and more typically at least two) vitamin selected from the group of vitamin B₁, vitamin B₃, vitamin B₅, vitamin B₆, vitamin B₇, vitamin B₉, and vitamin B₁₂, those in which vitamin B₃, is combined with at least one (and more typically at least two) vitamin selected from the group of vitamin B₂, vitamin B₁, vitamin B₅, vitamin B₆, vitamin B₇, vitamin B₉, and vitamin B₁₂, those in which vitamin B₅, is combined with at least one (and more typically at least two) vitamin selected from the group of vitamin B₂, vitamin B₃, vitamin B₁, vitamin B₆, vitamin B₇, vitamin B₉, and vitamin B₁₂, those in which vitamin B₆, is combined with at least one (and more typically at least two) vitamin selected from the group of vitamin B₂, vitamin B₃, vitamin B₅, vitamin B₁, vitamin B₇, vitamin B₉, and vitamin B₁₂, those in which vitamin B₇, is combined with at least one (and more typically at least two) vitamin selected from the group of vitamin B₂, vitamin B₃, vitamin B₅, vitamin B₆, vitamin B₁, vitamin B₉, and vitamin B₁₂, those in which vitamin B₉, is combined with at least one (and more typically at least two) vitamin selected from the group of vitamin B₂, vitamin B₃, vitamin B₅, vitamin B₆, vitamin B₇, vitamin B₁, and vitamin B₁₂, and those in which vitamin B₁₂, is combined with at least one (and more typically at least two) vitamin selected from the group of vitamin B₂, vitamin B₃, vitamin B₅, vitamin B₆, vitamin B₇, vitamin B₉, and vitamin B₁.

For example, vitamin B₆ may be administered as pyridoxine, pyridoxine 5′-phosphate, pyridoxal, pyridoxal 5′-phosphate, pyridoxamine, pyridoxamine 5′-phosphate, and/or 4-pyridoxic acid, while vitamin B₁₂ may be provided as a cobalamin such as a cyanocobalamin, hydroxycobalamin, 5-deoxyadenosylcobalamin, and/or as adenosylcobalamin. Similarly, vitamin B₂ may be provided as riboflavin, riboflavinphosphate, etc. With respect to suitable vitamin B₁ compounds, it is noted that thiamine, thiamin monophosphate, thiamin diphosphate, thiamin triphosphate, and adenosine thiamin triphosphate are suitable for use herein, and that vitamin B₃ can be provided as niacin or niacin derivative (e.g., niacinamide, niacin phosphate, nicotinamide riboside, etc.). With respect to vitamin B₅ it is generally preferred that the vitamin B₅ is provided as a mineral (e.g., calcium) salt or as pantothenol or panthenol, while vitamin B₉ is preferably administered as dihydrofolate, tetrahydrofolate, and/or methylenetetrahydrofolate.

Preferred relative (weight or molar) ratios of the vitamin B compounds are similar to those shown in Table 1. However, it should be noted that the molar ratios may also be modified such that one or two of the vitamin B compounds are in at least 2-fold, more preferably at least 5-fold, and even more preferably at least 10-fold molar excess over the remaining vitamin B compound(s). Regardless of the exact weights and ratios of the vitamin B compounds, it is generally preferred that each of the vitamin B compounds will be within 0.01-fold to 100-fold of the RDA (recommended daily allowance) for the respective vitamin B compound, and more typically within 0.1-fod to 10-fold of RDA.

Of course, it should be appreciated that contemplated compositions presented herein may also comprise vitamins other than the vitamin B compounds, which may be present in addition to or in lieu of the vitamin B compounds. Consequently, contemplated compositions will also include vitamins that are hydrophilic (e.g., vitamin C its derivatives) and/or those that are lipophilic (e.g. vitamin A, vitamin D, vitamin E, vitamin K, and their derivatives). Most typically, these non-vitamin B compounds will be present in quantities between 0.01-fold to 100-fold of the RDA. Where such non-vitamin B compounds are included to at least one vitamin B compound, it is preferred that the addition of the non-vitamin B compound is such that the non-vitamin B compound increases SIRT activity (additively or synergistically).

Depending of the particular formulation and/or use, it should be appreciated that the vitamin compound(s) may be provided in physiologically active form, in a prodrug forms, or in a chemical derivative (e.g., where the derivative has increased bioavailability, increased solubility, reduced rate of metabolism, increased specificity towards a target organ or tissue, etc.). In less preferred aspects, metabolites of the vitamin compounds are also contemplated.

Still further additional ingredients to contemplated compositions include those that are already known to activate SIRT. For example, suitable additional ingredients include one or more flavones, stilbenes (and particularly resveratrol), catechins, flavanones, and/or anthocyanidins, which may be added as purified or isolated compounds, or may be present in the form of an extract or other plant preparation. Particularly preferred flavones include apigenin, luteolin, tangeritin, chrysin, 6-hydroxyflavone, baicalein, and scutellarein, and preferred stilbenes include substituted (cis- and trans-) stilbenes, and particularly hydroxylated stilbenes (e.g., resveratrol, quercetin etc.). Suitable flavanones include butin, eriodictyol, hesperetin, hesperidin, homoeriodictyol, isosakuranetin, naringenin, naringin, pinocembrin, poncirin, sakuranetin, sakuranin, and sterubin, and suitable catechins especially include green tea catechins (e.g., catechin, epicatechin, gallocatechin, epigallocatechin, and respective gallates, in (+) and (−) conformation). Contemplated anthocyanidins include aurantinidin, cyanidin, delphinidin, europinidin, luteolinidin, pelargonidin, malvidin, peonidin, petunidin, and rosinidin. Similar to the addition of non-vitamin B compounds, and regardless of the exact weights and ratios of the vitamin B compounds (or non-vitamin B compounds), it is generally preferred that each of the additional ingredients will be within 0.01-fold to 100-fold of the RDA (recommended daily allowance) for the respective additional ingredient, and more typically within 0.1-fod to 10-fold of RDA.

It is typically preferred that the compositions according to the inventive subject matter are formulated for oral delivery, and all known formulations for oral delivery are deemed suitable for use herein. For example, oral formulations include tablets, dragees, capsules, powders, aqueous or non-aqueous solutions or suspensions, syrup, etc. Most typically, such formulations will include at least one pharmaceutically or nutraceutically acceptable carrier and are typically prepared to allow administration of a recommended daily dosage in a single dosage unit form. Alternatively, where desired, the dosage unit may also be chosen such that multiple dosage units per day will provide the recommended daily dosage.

Alternatively, contemplated compositions may also be included in already known oral formulations. Consequently, contemplated formulations include multi-vitamin preparations and all known preparations are deemed suitable for use herein.

Moreover, contemplated compositions may also be included into an edible carrier to so increase actual or perceived nutritional value of the edible carrier. Most preferably, such edible carrier is in a ready-to-consume format and may be an energy drink, a bottled water product, a carbonated drink, etc., or a snack bar, a cereal, a confectionary item, a plant fiber-containing product etc. In less preferred aspects, parenteral administration is contemplated and preferably includes injection, transmucosal delivery, and sublingual administration.

Regardless of the particular ingredients and formulation, it should be noted that the increase in SIRT activity using contemplated compositions may be verified in numerous manners well known in the art. Most typically, SIRT activity is measured using commercially available test kits and cells obtained from a mammal or cell culture. Thus, test results will be available from human (or other mammalian) subjects as well as from cell cultures. For example, suitable tests include those in which SIRT1 deacetylase is quantified, for example, via antibodies (e.g., using test kit by Abnova GmbH, Boxbergring 107, 69126 Heidelberg, Germany) or via coupled protease activity (e.g., using test kit from MBL International, 4 H Constitution Way, Woburn, Mass. 01801). Thus, a test result may be obtained (by the provider of the composition or other party, including contract test laboratory) directly by performing the SIRT activity test using volunteer samples, or indirectly by having a test performed in a contract or otherwise affiliated laboratory, and even by having an independent and unaffiliated third party perform the test and publish the test result. Especially contemplated test results will include those in which increase in SIRT activity is reported as a function administration of the composition to a mammal (or other animal) or cells. Such report preferably provides qualitative information on the amount of the composition used and/or the increase in SIRT activity achieved. Most typically (but not necessarily), the composition used for the test result has the same or similar ingredients than the composition that is marketed or otherwise provided to a consumer.

Thus, in especially preferred aspects of the inventive subject matter, the compositions contemplated herein will be provided to a consumer (typically the user) in association with the test result to inform or suggest to the consumer that the composition is effective to increase SIRT activity. The term “in association with” is therefore meant to include any activity that logically (and most preferably also physically) couples the composition with the test result. For example, logical coupling includes displaying, printing, or otherwise providing information of the test result while making reference to the composition (e.g., displaying the test result and the composition). More preferably, however, the test result is physically associated with the composition. For example, such physical association may be performed by printing the test result on the container or packaging that holds the composition.

Viewed from a different perspective, various methods of increasing SIRT activity in mammalian cells are also contemplated where cells are exposed to a (preferably synergistic) combination of vitamin B compounds in an amount effective to increase SIRT activity in the mammal. As noted above, it is especially preferred that the synergistic combination includes at least two of vitamin B₆, vitamin B₁₂, and vitamin B₂, and more typically vitamin B₆, vitamin B₁₂, and vitamin B₂. Where desired, the synergistic combination may also include one or more of vitamin B₁, vitamin B₅, and vitamin B₉. regarding the specific quantities of the compounds in the combinations contemplated for such methods, the same considerations as provided above apply.

Therefore, it should be appreciated that numerous conditions, symptoms, and/or diseases could be impacted (and most preferably improved or even treated) by administration of contemplated compositions, where such condition, symptom, and/or disease is associated with SIRT expression. For example, contemplated compositions could be used to increase SIRT expression that is reduced due to aging or senescence. On the other hand, various conditions and diseases are known to be associated with expression of Nf-kB, which is in turn influenced by expression of SIRT. Still further, other examples of conditions that can be modulated by SIRT expression include muscle-waste, inflammation, senescence, activation and/or differentiation of stem cells, and endothelial dysfunction.

Thus, specific embodiments and applications of methods of SIRT activation have been disclosed. It should be apparent, however, to those skilled in the art that many more modifications besides those already described are possible without departing from the inventive concepts herein. The inventive subject matter, therefore, is not to be restricted except in the spirit of the appended claims.

What is claimed is:

1. A method of providing a composition that increases SIRT activity in a mammal, comprising:
formulating a composition that includes at least one vitamin B compound selected from the group consisting of vitamin B₆, vitamin B₁₂, and vitamin B₂ in an amount effective to increase SIRT activity in the mammal;
obtaining a test result that indicates that the at least one vitamin B compound increases SIRT activity in the mammal; and
providing the composition in association with the test result.

2. (canceled)

3. The method of claim 1 wherein the composition comprises vitamin B₆ and vitamin B₁₂.

4. The method of claim 1 wherein the composition comprises vitamin B₆, vitamin B₁₂, and vitamin B₂.

5. The method of claim 4 wherein the vitamin B₆, vitamin B₁₂, and vitamin B₂ are present in a synergistic amount.

6. The method of claim 1 wherein the composition further comprises at least one of a vitamin B₁ compound, a vitamin B₅ compound, and a vitamin B₉ compound.

7. The method of claim 1 wherein the composition further comprises at least one of a flavone, a stilbene, a flavanone, and an anthocyanidin, and optionally further comprises a vitamin other than the vitamin B compound.

8. The method of claim 1 wherein the vitamin B₆ is present in an amount of between 60 and 600 mg, wherein the vitamin B₁₂ is present in an amount of between 30 and 300 mcg, and wherein the vitamin B₂ is present in an amount of between 30 and 300 mg.

9. The method of claim 6 wherein the vitamin B₁ is present in an amount of between 30 and 300 mg, wherein the vitamin B₅ is present in an amount of between 30 and 300 mg, and wherein the vitamin B₉ is present in an amount of between 3 and 30 mg.

10. The method of claim 6 wherein the vitamin B₆ is present in an amount of about 220 mg, wherein the vitamin B₁₂ is present in an amount of about 90 meg, wherein the vitamin B₂ is present in an amount of about 100 mg, wherein the vitamin B₁ is present in an amount of about 100 mg, wherein the vitamin B₅ is present in an amount of about 100 mg, and wherein the vitamin B₉ is present in an amount of about 15 mg.

11. The method of claim 1 wherein the test result is a test result obtained from a human test subject.

12. The method of claim 1 wherein the increase SIRT activity is at least 80% over untreated control.

13. A method of providing a composition that increases SIRT activity in a mammal, comprising:
formulating a composition that includes at least two vitamin compounds in an amount effective to synergistically increase SIRT activity in the mammal;
obtaining a test result that indicates that the at least two vitamin compounds synergistically increase SIRT activity in the mammal; and
providing the composition in association with the test result.

14. A method of increasing SIRT activity in a mammalian cell, comprising exposing the cell to a synergistic combination of vitamin B compounds in an amount effective to increase SIRT activity in the mammal.

15. The method of claim 14 wherein the synergistic combination of vitamin B compounds comprises vitamin B₆, vitamin B₁₂, and vitamin B₂.

16. The method of claim 14 wherein the wherein the synergistic combination further comprises at least one of vitamin B₁, vitamin B₅, and vitamin B₉.

17. The method of claim 14 wherein the wherein the synergistic combination further comprises at least two of vitamin B₁, vitamin B₅, and vitamin B₉.

18. The method of claim 15 wherein the vitamin B₆ is present in an amount of between 60 and 600 mg, wherein the vitamin B₁₂ is present in an amount of between 30 and 300 mcg, and wherein the vitamin B₂ is present in an amount of between 30 and 300 mg.

19. The method of claim 16 wherein the vitamin B₁ is present in an amount of between 30 and 300 mg, wherein the vitamin B₅ is present in an amount of between 30 and 300 mg, and wherein the vitamin B₉ is present in an amount of between 3 and 30 mg.

20. The method of claim 16 wherein the vitamin B₆ is present in an amount of about 220 mg, wherein the vitamin B₁₂ is present in an amount of about 90 mcg, wherein the vitamin B₂ is present in an amount of about 100 mg, wherein the vitamin B₁ is present in an amount of about 100 mg, wherein the vitamin B₅ is present in an amount of about 100 mg, and wherein the vitamin B₉ is present in an amount of about 15 mg.
